(19) United States
(12) Patent Application Publication
(10) Pub. No.: US 2011/0009614 A1
(43) Pub. Date: Jan. 13, 2011

(54) PROCESSES AND REACTOR SYSTEMS FOR CONVERTING SUGARS AND SUGAR ALCOHOLS

(76) Inventors: Paul George Blommel, Oregon, WI (US); Elizabeth M. Woods, Middleton, WI (US); Michael J. Werner, Portage, WI (US); Aaron James Imrie, Verona, WI (US); Randy D. Cortright, Madison, WI (US)

Correspondence Address: Quarles & Brady LLP, PO Box 2113, Madison, WI, 53701-2113, US

(21) Appl. No.: 12/827,827

(22) Filed: Jun. 30, 2010

Related U.S. Application Data
(60) Provisional application No. 61/221,942, filed on Jun. 30, 2009.

(51) Int. Cl. C07H 1/00; B01J 38/10; B01J 38/48; B01J 38/52

(52) U.S. Cl. 536/124; 502/53; 502/22; 502/33

(57) ABSTRACT

Processes and reactor systems are provided for the conversion of sugars to sugar alcohols using a hydrogenation catalyst, which includes apparatus and method for in-line regeneration of the hydrogenation catalyst to remove carbonaceous deposits.

### CROSS-REFERENCE TO RELATED APPLICATION

This application claims the benefit of U.S. Provisional Application No. 61/221,942 filed on Jun. 30, 2009.

### STATEMENT REGARDING FEDERALLY SPONSORED RESEARCH OR DEVELOPMENT

Not Applicable.

### BACKGROUND OF THE INVENTION

Aqueous-Phase Reforming (APR) is a catalytic reforming process that generates hydrogen and hydrocarbons from oxygenated compounds derived from a wide array of biomass, including glycerol, sugars, sugar alcohols, etc. Various APR methods and techniques are described in U.S. Pat. Nos. 6,699,457; 6,964,757; 6,964,758; and 7,618,612 (all to Cortright et al., and entitled “Low-Temperature Hydrogen Production from Oxygenated Hydrocarbons”); U.S. Pat. No. 6,953,873 (to Cortright et al., and entitled “Low-Temperature Hydrocarbon Production from Oxygenated Hydrocarbons”); U.S. Patent Application Ser. No. 2008/0025903 (to Cortright, and entitled “Methods and Systems for Generating Polyols”); U.S. Patent Application Ser. Nos. 2008/0216391; 2008/0300434; and 2008/0300435 (all to Cortright and Blommel, and entitled “Synthesis of Liquid Fuels and Chemicals from Oxygenated Hydrocarbons”); U.S. Patent Application Ser. No. 2009/0211942 (to Cortright, and entitled “Catalysts and Methods for Reforming Oxygenated Compounds”); U.S. Patent Application Ser. No. 2010/0076233 (to Cortright et al., and entitled “Synthesis of Liquid Fuels from Biomass”); International Patent Application No. PCT/US2008/056330 (to Cortright and Blommel, and entitled “Synthesis of Liquid Fuels and Chemicals from Oxygenated Hydrocarbons”); and commonly owned co-pending International Patent Application No. PCT/US2006/048030 (to Cortright et al., and entitled “Catalyst and Methods for Reforming Oxygenated Compounds”), all of which are incorporated herein by reference.

In certain applications, it may be beneficial for sugars to be hydrogenated to increase their thermal stability prior to their use as a feed for APR. At temperatures compatible with APR, sugars are susceptible to thermal degradation, which leads to byproduct formation, catalyst fouling, and, ultimately, shortened time between catalyst regenerations. This problem is avoided by reacting sugars with hydrogen to form polyols or sugar alcohols that are more thermally stable.

The hydrogenation of sucrose is shown in FIG. 1. The α-1,2 glycosidic bond present in sucrose requires an initial hydrolysis step before either monomer can be hydrogenated. After hydrolysis, glucose is selectively hydrogenated to sorbitol, while fructose is hydrogenated to a mixture of sorbitol and mannitol.

The hydrogenation process described above causes catalyst fouling due to the build-up of carbonaceous deposits on the surface of the hydrogenation catalyst over time. As these deposits accumulate, access to the catalytic sites on the surface becomes restricted and the catalyst's performance declines, resulting in lower conversion and yields of polyol products. Changing the hydrogenation catalyst frequently is time consuming and expensive, especially for in-line continuous processes. As a result, most industrial applications involve a batch or semi-continuous process. Therefore, a process for regenerating the hydrogenation catalyst to allow for continued use would be beneficial.

There exists a need for a method of regenerating a hydrogenation catalyst used in the catalytic conversion of biomass to biofuels. Processes that can take place in the same reactor system as the hydrogenation would be especially advantageous.

### DESCRIPTION OF THE DRAWINGS

FIG. 1 illustrates the hydrogenation of sucrose to form polyols and sugar alcohols.

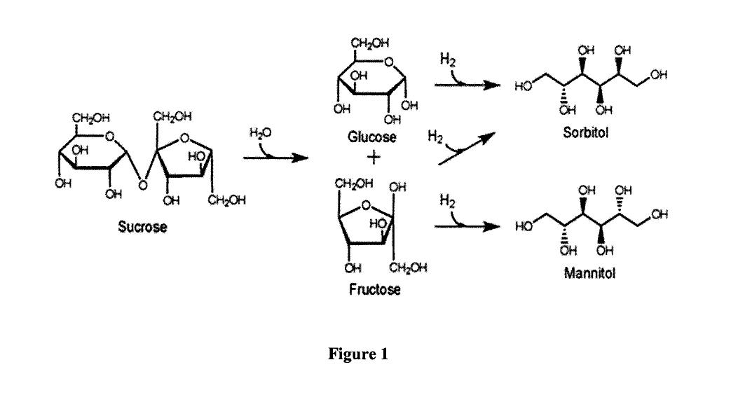

FIG. 2 is a flow diagram illustrating a reactor system for the present invention.

FIG. 3 is a flow diagram illustrating a shell & tube reactor system for the present invention.

FIG. 4 is a graph illustrating methane and ethane content of the purge gas during the hydrogenation catalyst regeneration.

FIG. 5 is a graph illustrating methane, ethane, propane, and butane content of the purge gas during the hydrogenation catalyst regeneration. Methane is the dominant species at all temperatures, but it evolves more rapidly at higher temperatures. The data shows that heavier hydrocarbons are removed more rapidly at lower temperatures.

FIG. 6 is a graph that compares the yield of polyols converted from sucrose before and after hydrogenation catalyst regeneration.

FIG. 7 is a graph that shows carbon removed over time during regeneration and the temperature profile of the reactor during regeneration.

### SUMMARY OF THE INVENTION

One aspect of the invention is a method for regenerating a hydrogenation catalyst. The method includes the steps or acts of providing a hydrogenation catalyst containing carbonaceous deposits, flushing the hydrogenation catalyst with a flushing medium, contacting the hydrogenation catalyst with hydrogen, maintaining a flow of hydrogen over the hydrogenation catalyst, adjusting the pressure on the hydrogenation catalyst to a regeneration pressure of about atmospheric pressure to about 3000 psig, and adjusting the temperature of the hydrogenation catalyst to a regeneration temperature in the range of about 250° C. to about 400° C., wherein carbonaceous deposits are removed from the hydrogenation catalyst and the hydrogenation catalyst is regenerated such that hydrogenation can be resumed.

In an exemplary embodiment of the method for regenerating a hydrogenation catalyst, the step of flushing the hydrogenation catalyst with the flushing medium is conducted at a flushing temperature below about 100° C.

In another exemplary embodiment of the method for regenerating a hydrogenation catalyst, the flushing medium is in the liquid phase.

In another exemplary embodiment of the method for regenerating a hydrogenation catalyst, the temperature of the hydrogenation catalyst is adjusted to the regeneration temperature at a rate of about 20° C. per hour to about 100° C. per hour.

In another exemplary embodiment of the method for regenerating a hydrogenation catalyst, the regeneration temperature is maintained for approximately eight hours.

In another exemplary embodiment of the method for regenerating a hydrogenation catalyst, the regeneration pressure is in the range of about 600 psig to about 1500 psig.

In another exemplary embodiment of the method for regenerating a hydrogenation catalyst, the method removes about 98% of the carbonaceous deposits from the hydrogenation catalyst.

In another exemplary embodiment of the method for regenerating a hydrogenation catalyst, the flushing medium is selected from the group consisting of water, an alcohol, a ketone, a cyclic ether, a water-soluble oxygenated hydrocarbon, and a combination of at least two of the foregoing.

In another exemplary embodiment of the method for regenerating a hydrogenation catalyst, the hydrogenation catalyst is flushed in the presence of hydrogen to maintain an oxygen-free environment.

In another exemplary embodiment of the method for regenerating a hydrogenation catalyst, the hydrogenation catalyst acted upon in the method includes a support and a catalytic member selected from the group consisting of Fe, Ru, Os, Ir, Co, Rh, Pt, Pd, Ni, Re, Cu, an alloy of at least two of the foregoing, and a combination of at least two of the foregoing.

In another exemplary embodiment of the method for regenerating a hydrogenation catalyst, the hydrogenation catalyst acted upon in the method further includes a second catalytic material selected from the group consisting of Ag, Au, Cr, Zn, Mn, Sn, Bi, Mo, W, B, P, an alloy of at least two of the foregoing, and a combination of at least two of the foregoing.

In another exemplary embodiment of the method for regenerating a hydrogenation catalyst, the support includes a member selected from the group consisting of a nitride, carbon, silica, alumina, zirconia, titania, vanadia, ceria, boron nitride, heteropolyacid, kieselguhr, hydroxyapatite, zinc oxide, chromia, and a combination of at least two of the foregoing.

In another exemplary embodiment of the method for regenerating a hydrogenation catalyst, the support is a carbon support and the hydrogenation catalyst is flushed in the presence of hydrogen to maintain an oxygen-free environment.

Another aspect of the invention is a method for hydrogenation of a sugar and in-line regeneration of a hydrogenation catalyst that contains carbonaceous deposits. The method includes the steps or acts of catalytically reacting in a liquid or vapor phase an aqueous feedstock solution comprising water and a sugar with hydrogen in the presence of the hydrogenation catalyst at a hydrogenation temperature and a hydrogenation pressure, replacing the aqueous solution with a flushing medium, contacting the hydrogenation catalyst with hydrogen, maintaining a flow of hydrogen over the hydrogenation catalyst, adjusting the pressure on the hydrogenation catalyst to a regeneration pressure in the range of about atmospheric pressure to about 3000 psig, adjusting the temperature of the hydrogenation catalyst to a regeneration temperature in the range of about 250° C. to about 400° C. and wherein the carbonaceous deposits are removed from the hydrogenation catalyst and the hydrogenation catalyst is regenerated such that hydrogenation can be resumed, returning the hydrogenation catalyst to the hydrogenation temperature and the hydrogenation pressure, and catalytically reacting the aqueous feedstock solution with hydrogen in the presence of the hydrogenation catalyst at the hydrogenation temperature and the hydrogenation pressure.

In an exemplary embodiment of the method for hydrogenation of a sugar and in-line regeneration of a hydrogenation catalyst that contains carbonaceous deposits, the step of flushing the hydrogenation catalyst with the flushing medium is conducted at a flushing temperature below about 100° C.

In an exemplary embodiment of the method for hydrogenation of a sugar and in-line regeneration of a hydrogenation catalyst that contains carbonaceous deposits, the flushing medium is in the liquid phase.

In another exemplary embodiment of the method for hydrogenation of a sugar and in-line regeneration of a hydrogenation catalyst that contains carbonaceous deposits, the temperature of the hydrogenation catalyst is adjusted to the regeneration temperature at a rate of about 20° C. per hour to about 100° C. per hour.

In another exemplary embodiment of the method for hydrogenation of a sugar and in-line regeneration of a hydrogenation catalyst that contains carbonaceous deposits, the regeneration temperature is maintained for approximately eight hours.

In another exemplary embodiment of the method for hydrogenation of a sugar and in-line regeneration of a hydrogenation catalyst that contains carbonaceous deposits, the regeneration pressure is in the range of about 600 psig to about 1500 psig.

In another exemplary embodiment of the method for hydrogenation of a sugar and in-line regeneration of a hydrogenation catalyst that contains carbonaceous deposits, about 98% of the carbonaceous deposits are removed from the hydrogenation catalyst.

In another exemplary embodiment of the method for hydrogenation of a sugar and in-line regeneration of a hydrogenation catalyst that contains carbonaceous deposits, the flushing medium is selected from the group consisting of water, an alcohol, a ketone, a cyclic ether, a water-soluble oxygenated hydrocarbon, and a combination of at least two of the foregoing.

In another exemplary embodiment of the method for hydrogenation of a sugar and in-line regeneration of a hydrogenation catalyst that contains carbonaceous deposits, the hydrogenation catalyst is flushed in the presence of hydrogen to maintain an oxygen-free environment.

In another exemplary embodiment of the method for hydrogenation of a sugar and in-line regeneration of a hydrogenation catalyst that contains carbonaceous deposits, the hydrogenation catalyst includes a support and a catalytic material selected from the group consisting of Fe, Ru, Os, Ir, Co, Rh, Pt, Pd, Ni, Re, Cu, an alloy of at least two of the foregoing, and a combination of at least two of the foregoing.

In another exemplary embodiment of the method for hydrogenation of a sugar and in-line regeneration of a hydrogenation catalyst that contains carbonaceous deposits, the hydrogenation catalyst further includes a second catalytic material selected from the group consisting of Ag, Au, Cr, Zn, Mn, Sn, Bi, Mo, W, B, P, an alloy of at least two of the foregoing, and a combination of at least two of the foregoing.

In another exemplary embodiment of the method for hydrogenation of a sugar and in-line regeneration of a hydrogenation catalyst that contains carbonaceous deposits, the support includes a member selected from the group consisting of a nitride, carbon, silica, alumina, zirconia, titania, vanadia, ceria, boron nitride, heteropolyacid, kieselguhr, hydroxyapatite, zinc oxide, chromia, and a combination of at least two of the foregoing.

In another exemplary embodiment of the method for hydrogenation of a sugar and in-line regeneration of a hydrogenation catalyst that contains carbonaceous deposits, the support is a carbon support and the hydrogenation catalyst is flushed in the presence of hydrogen to maintain an oxygen-free environment.

### DESCRIPTION OF THE INVENTION

The present invention relates to methods and reactor systems for converting sugars to sugar alcohols. The process includes a method for the in-line regeneration of hydrogenation catalysts. The hydrogenation catalyst can be regenerated to remove the carbonaceous deposits and regain activity. Hydrogenation catalysts can be regenerated in the same reactor vessel used to hydrogenate the starting sugar into sugar alcohols. The known hydrogenation structure is modified to accomplish regeneration of the hydrogenation catalyst. Preferably, the reactor system is modified to include an inlet for a flushing medium.

Hydrogenation Conditions

In general, hydrogenation reactions should be carried out at a temperature at which the thermodynamics of the proposed reaction are favorable. Hydrogenation temperature and pressure conditions can be selected to maintain either a liquid or vapor phase reaction. Generally, a suitable hydrogenation temperature is in the range of about 80° C. to about 180° C., with hydrogenation pressure in the range of about 100 psig to about 3000 psig. Within this range, higher pressures lead to higher reaction rates and potentially slower catalyst deactivation as hydrogen solubility increases in the liquid phase, however, the pressure may be limited by equipment and operating costs. As a result, the desired operating pressure is often determined by weighing different factors and is generally chosen to result in the most economically favorable process.

Feedstock Solution

A suitable feedstock solution includes water-soluble sugars derived from biomass. As used herein, the term “biomass” refers to, without limitation, organic materials produced by plants (such as leaves, roots, seeds and stalks), and microbial and animal metabolic wastes. Common biomass sources include: (1) agricultural wastes, such as corn stalks, straw, seed hulls, sugarcane leavings, bagasse, nutshells, and manure from cattle, poultry, and hogs; (2) wood materials, such as wood or bark, sawdust, timber slash, and mill scrap; (3) municipal waste, such as waste paper and yard clippings; and (4) energy crops, such as poplars, willows, switch grass, alfalfa, prairie bluestream, corn, soybean, and the like. The feedstock can be fabricated from biomass by any means now known or developed in the future, or can be simply byproducts of other processes.

The sugars can also be derived from wheat, corn, sugar beets, sugar cane, or molasses. The sugar is combined with water to provide an aqueous feedstock solution having a concentration effective for hydrogenating the sugar. Generally, a suitable concentration is in the range of about 5% to about 70%, with a range of about 40% to 70% more common in industrial applications.

Hydrogenation Technology

The following hydrogenation reactor system and process is provided for context. Hydrogenation reactions can be carried out in any reactor of suitable design, including continuous-flow, batch, semi-batch or multi-system reactors, without limitation as to design, size, geometry, flow rates, etc. The reactor system can also use a fluidized catalytic bed system, a swing bed system, a fixed bed system, a moving bed system, or a combination of the above. Preferably, the present invention is practiced utilizing a continuous-flow system at steady-state equilibrium.

For many multiphase reactions, the preferred reactor type is a trickle bed reactor in which the gas and liquid feeds are introduced at the top of the reactor and then allowed to flow downward over a fixed bed of catalyst. The advantages of the trickle bed reactor include simple mechanical design, simplified operation and potentially simplified catalyst development. The main design challenges are ensuring that the heat and mass transfer requirements of the reaction are met. The main operational challenges for trickle bed reactors are: uniformly loading the catalyst, uniformly introducing the gas and liquid feeds, and avoiding bypassing of some of the catalyst due to channeling of the reactants as they flow through the reactor.

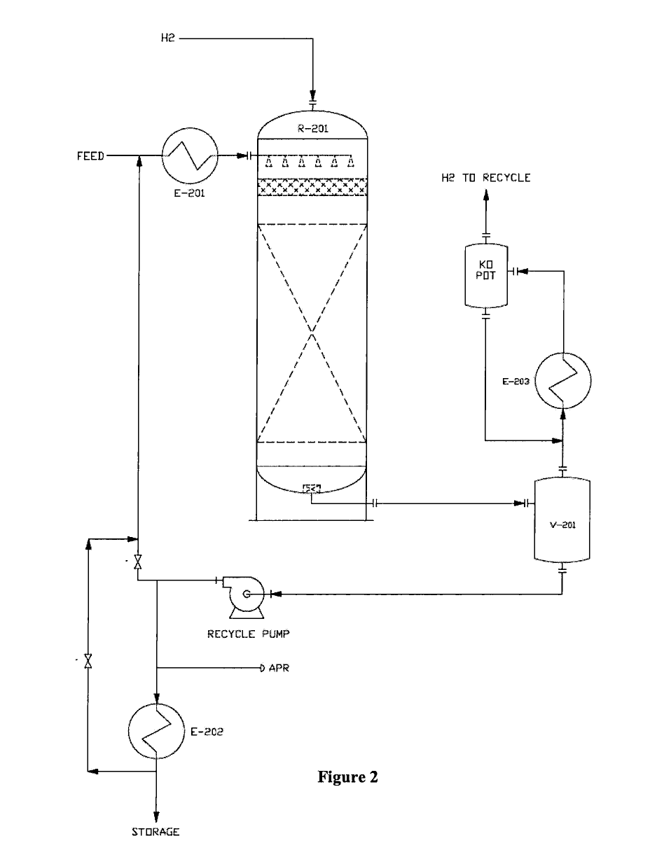

Illustrated in FIG. 2 is a trickle bed reactor employed in practicing the present invention. Liquid and hydrogen feeds are reacted across a reactor bed that includes a catalyst on a support, such as ruthenium supported on carbon. For sucrose, the hydrogenation must be preceded by hydrolysis. Hydrogen solubility is limited in sugar and polyol solutions and is a strong function of the gas phase hydrogen partial pressure. Thus, the reaction can be limited by the amount of hydrogen available in the aqueous phase, and high operating pressures are desirable to increase aqueous hydrogen concentration. In this application, the hydrogenation step can operate between about 100 psig and about 3000 psig to achieve the hydrogen partial pressure required for hydrogenation while avoiding the capital and operating costs that would be required by higher pressure operation. The temperature of the hydrogenation system will vary depending on the catalyst, feedstock, and pressure. When a ruthenium hydrogenation catalyst is employed in applications involving a sucrose feedstock, the hydrogenation system can operate between about 80° C. and about 180° C.

The primary alternative design to a trickle bed reactor is a slurry reactor. While a trickle bed reactor is loaded with an immobile catalyst, a slurry reactor contains a flowing mixture of reactants, products, and fine catalyst particles. Keeping a uniform mixture throughout the reactor vessel requires active mixing either from a mixer or a pump. In addition, to withdraw product the catalyst particles must be separated from the product and unreacted feed by filtration, settling, centrifuging or some other means. Finally, in contrast to the trickle bed reactor catalyst, the catalyst in a slurry reactor must be highly resistant to attrition due to the mixer. The advantages of a slurry reactor are mainly that the active mixing might enable higher heat and mass transfer rates per unit of reactor volume.

Hydrogenation Operations

In a continuous flow system, the reactor system includes a hydrogenation reactor vessel adapted to receive an aqueous feedstock solution and a method for controlling the temperature of the reactor, such as a heat exchanger. The reactor vessel preferably includes an outlet adapted to remove the product stream from the reactor vessel. The reactor system can also include additional inlets which allow supplemental materials, such as hydrogen or a flushing medium, to be introduced into the reactor system.

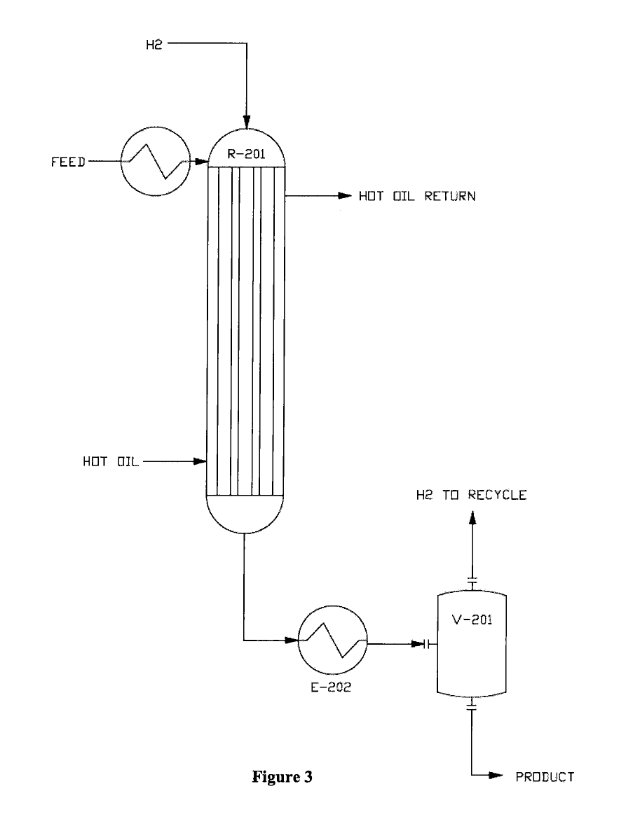

FIG. 3 illustrates an example hydrogenation reaction. Feed is delivered to the hydrogenation section from a feed preparation area and then brought up to the desired temperature by exchange with a circulating hot oil medium in the hydrogenation feed preheater E-201. The temperature at this point is between about 80° C. and about 140° C. The feed is then directed into the hydrogenation reactor R-201 and distributed across nine tubes in a shell and tube reactor which contain the hydrogenation catalyst. In a preferred embodiment, the hydrogenation catalyst is a ruthenium based catalyst. Recycled and fresh hydrogen are also brought into the reactor and distributed between the tubes. As the feeds pass though the reactor, water and hydrogen are consumed, glucose and fructose are present as intermediates, and sorbitol and mannitol are formed as the final reaction products. The reaction is exothermic, and the maximum possible temperature rise, the adiabatic temperature rise, is a function of the feedstock concentration. The adiabatic temperature rise for a 50 wt % sucrose solution is estimated to be about 90° C.

To maintain the desired operating temperature, which is generally in the range of about 80° C. to about 180° C., a hot oil system is employed on the shell side of the shell and tube hydrogenation reactor. The hot oil system, by its unique design, allows either heat removal or heat addition to the system, depending on the needs of the process. To provide cooling, a portion of the circulating hot oil is passed through a cooling water exchanger prior to reentering the reactor, with the amount routed through the cooler dependent on the required cooling duty. To provide heat, additional hot oil is routed into the circulation system from the high temperature hot oil reservoir.

Hydrogenation reactions take place in the presence of a hydrogenation catalyst, either a homogenous catalyst or heterogeneous catalyst that includes a support. Suitable hydrogenation catalysts, supports, and reaction conditions are described in detail in PCT/US2008/056330, previously incorporated by reference. Other processes known for hydrogenating sugars, furfurals, carboxylic acids, ketones, and furans to their corresponding alcohol form, include those disclosed by: B. S. Kwak et al. (WO2006/093364A1 and WO 2005/021475A1), involving the preparation of sugar alditols from monosaccharides by hydrogenation over a ruthenium catalyst, incorporated herein by reference; and Elliot et al. (U.S. Pat. Nos. 6,253,797 and 6,570,043), disclosing the use of a nickel and rhenium free ruthenium catalyst on a more than 75% rutile titania support to convert sugars to sugar alcohols, also incorporated herein by reference. Other suitable ruthenium catalysts are described by Arndt et al. in published U.S. patent application 2006/0009661 (filed Dec. 3, 2003), and Arena in U.S. Pat. Nos. 4,380,679 (filed Apr. 12, 1982), 4,380,680 (filed May 21, 1982), 4,503,274 (filed Aug. 8, 1983), 4,382,150 (filed Jan. 19, 1982), and 4,487,980 (filed Apr. 29, 1983), all incorporated herein by reference.

Other systems include those described by Arena in U.S. Pat. No. 4,401,823 (filed May 18, 1981) directed to the use of a carbonaceous pyropolymer catalyst containing transition metals (such as chromium, molybdenum, tungsten, rhenium, manganese, copper, cadmium) or Group VIII metals (such as iron, cobalt, nickel, platinum, palladium, rhodium, ruthenium, iridium and osmium) to produce alcohols, acids, ketones, and ethers from polyhydroxylated compounds, such as sugars and sugar alcohols, and U.S. Pat. No. 4,496,780 (filed Jun. 22, 1983) directed to the use of a catalyst system having a Group VIII noble metal on a solid support with an alkaline earth metal oxide to produce glycerol, ethylene glycol and 1,2-propanediol from carbohydrates, each incorporated herein by reference. Another system includes that described by Dubeck et al. in U.S. Pat. No. 4,476,331 (filed Sep. 6, 1983) directed to the use of a sulfide-modified ruthenium catalyst to produce ethylene glycol and propylene glycol from larger polyhydric alcohols, such as sorbitol, also incorporated herein by reference. Other systems include those described by Saxena et al., “Effect of Catalyst Constituents on (Ni, Mo, and Cu)/Kieselguhr-Catalyzed Sucrose Hydrogenolysis,” Ind. Eng. Chem. Res. 44, 1466-1473 (2005), describing the use of Ni, W, and Cu on a kieselguhr support, incorporated herein by reference.

The hydrogenation catalyst generally includes Cu, Re, Ni, Fe, Co, Ru, Pd, Pt, Os, Ir, and alloys or combinations of at least two of the foregoing, either alone or with promoters such as W, Mo, Au, Ag, Cr, Zn, Mn, Sn, B, P, Bi, and alloys or combinations of at least two of the foregoing. The hydrogenation catalyst may also include any one of the supports further described below, and depending on the desired functionality of the catalyst. Other effective hydrogenation catalyst materials include either supported nickel or ruthenium modified with rhenium. In general, the hydrogenation reaction is carried out at hydrogenation temperatures of between about 80° C. to 180° C. depending on the feedstock and pressure, and with hydrogenation pressures in the range of about 100 psig to about 3000 psig.

The hydrogenation catalyst may also include a supported Group VIII metal catalyst and a metal sponge material, such as a sponge nickel catalyst. Activated sponge nickel catalysts (e.g., Raney nickel) are a well-known class of materials effective for various hydrogenation reactions. One type of sponge nickel catalyst is the type A7063 catalyst available from Activated Metals and Chemicals, Inc., Sevierville, Tenn. The type A7063 catalyst is a molybdenum promoted catalyst, typically containing approximately 1.5% molybdenum and 85% nickel. The use of the sponge nickel catalyst with a feedstock comprising xylose and dextrose is described by M. L. Cunningham et al. in U.S. Pat. No. 6,498,248, filed Sep. 9, 1999, incorporated herein by reference. The use of a Raney nickel catalyst with hydrolyzed corn starch is also described in U.S. Pat. No. 4,694,113, filed Jun. 4, 1986, and incorporated herein by reference.

The preparation of suitable Raney nickel hydrogenation catalysts is described by A. Yoshino et al. in published U.S. patent application 2004/0143024, filed Nov. 7, 2003, incorporated herein by reference. The Raney nickel catalyst may be prepared by treating an alloy of approximately equal amounts by weight of nickel and aluminum with an aqueous alkali solution, e.g., containing about 25 wt. % of sodium hydroxide. The aluminum is selectively dissolved by the aqueous alkali solution leaving particles having a sponge construction and composed predominantly of nickel with a minor amount of aluminum. Promoter metals, such as molybdenum or chromium, may be also included in the initial alloy in an amount such that about 1-2 wt. % remains in the sponge nickel catalyst.

In another embodiment, the hydrogenation catalyst is prepared by impregnating a suitable support material with a solution of ruthenium (III) nitrosylnitrate or ruthenium (III) chloride in water to form a solid that is then dried for 13 hours at 120° C. in a rotary ball oven (residual water content is less than 1% by weight). The solid is then reduced at atmospheric pressure in a hydrogen stream at 300° C. (uncalcined) or 400° C. (calcined) in the rotary ball furnace for 4 hours. After cooling and rendering inert with nitrogen, the catalyst may then be passivated by passing over 5% by volume of oxygen in nitrogen for a period of 120 minutes.

In yet another embodiment, the hydrogenation reaction is performed using a catalyst comprising a nickel-rhenium catalyst or a tungsten-modified nickel catalyst. One example of a suitable hydrogenation catalyst is the carbon-supported nickel-rhenium catalyst composition disclosed by Werpy et al. in U.S. Pat. No. 7,038,094, filed Sep. 30, 2003, and incorporated herein by reference.

A preferred hydrogenation catalyst can be prepared by adding an aqueous solution of dissolved ruthenium nitrosyl nitrate to a carbon catalyst support (OLC Plus, Calgon) with particle sizes restricted to those that were maintained on a 40 mesh screen after passing through an 18 mesh screen to a target loading of 2.5% ruthenium. Water can be added in excess of the pore volume and evaporated off under vacuum until the catalyst is free flowing. The catalyst can then be dried overnight at about 100° C. in a vacuum oven.

Hydrogenation Catalyst Reduction

The catalyst loaded in the hydrogenation reactor must be reduced in order to be in the active state. During catalyst production, the catalyst can be reduced and, in certain applications, then passivated with low levels of oxygen to stabilize the catalyst when exposed to air. The purpose of the reduction step is to transform any oxidized catalyst (e.g., ruthenium) into a fully reduced state.

Hydrogenation Catalyst Regeneration.

During hydrogenation, carbonaceous deposits build up on the hydrogenation catalyst surface. These deposits form through minor side reactions of the hydrogenation feed and products. As these deposits accumulate, access to the catalytic sites on the surface becomes restricted and the hydrogenation performance declines, resulting in lower conversion and yields of polyol products. To compensate for a loss in catalyst activity, the reaction temperature is increased. The temperature is generally only raised to about 150° C., and in no event greater than about 180° C. At temperatures above about 150° C. side reaction rates increase and catalyst deactivation markedly increases.

The first step in regenerating the hydrogenation catalyst is to flush the hydrogenation catalyst with a suitable flushing medium. The flushing medium can be any medium capable of washing unreacted species from the catalyst and reactor system. Such flushing medium may include any one of several gases other than oxygen (such as hydrogen, nitrogen, helium, etc.), and liquid media, such as water, alcohols, ketones, cyclic ethers, or other oxygenated hydrocarbons, whether alone or in combination with any of the foregoing, and which does not include materials known to be poisons for the catalyst in use (e.g., sulfur). The flushing step should be conducted at a temperature that does not cause a liquid phase flushing medium or the unreacted species to change to the gaseous phase. In one embodiment, the temperature is maintained below about 100° C. during the flushing step.

After completing the flush step, the flow of the flushing medium is terminated, and a constant flow of hydrogen is maintained. The temperature in the reactor is increased at a rate of no more than about 100° C./hour. At temperatures below 200° C., C—O and C—C linkages in the carbonaceous deposits are broken and C₂-C₆ alkanes, volatile oxygenates, and water are released from the catalyst. As temperatures continue to rise toward about 400° C., C—C bond hydrogenolysis predominates.

The carbon number of the material released decreases with increasing temperature. During the catalyst regeneration, light paraffins such as methane, ethane, and propane are emitted as a regeneration stream as the carbonaceous deposits are removed from the catalyst. While methane makes up the largest fraction of the carbon removed at all temperatures, significant levels of larger paraffins are evolved as well. The composition of the larger paraffins gradually shifts from longer chain components such as pentane and hexane to shorter chain paraffins, such as ethane and methane, as the temperatures increase and the regeneration progresses.

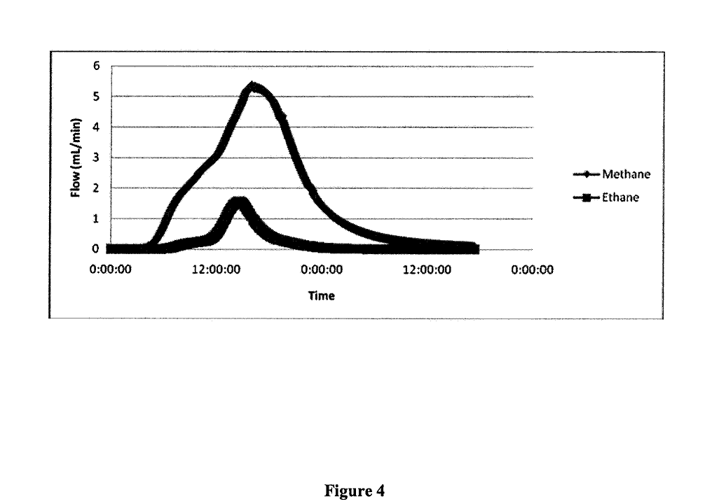

One method of monitoring the regeneration stream is using a gas chromatogram, such as an SRI 9610C GC with thermal conductivity and flame ionizing detectors in series using a molecular sieve column and a silica gel column in column switching arrangement for component separation. The product profile over time as reported by the SRI GC is shown in FIG. 4 and illustrates the typical trend of an inverse relationship between paraffin abundance and carbon number. Based on this trend, to obtain a maximum return of performance, the regeneration is continued until the methane content of the regeneration stream is below 0.3% by volume. However, a general increase in activity can also be seen with substantially greater residual paraffin content. The catalyst is considered completely regenerated when sufficient carbonaceous deposits have been removed such that hydrogenation can be resumed. This generally occurs when the methane given off during the hydrogenation catalyst regeneration decreases to an insignificant amount. In a preferred embodiment, the hydrogenation catalyst is considered regenerated when the amount of methane in the hydrogen catalyst regeneration environment is less than 4%, more preferably less than 2%, and most preferably less than 0.3%.

The accumulation of paraffins during regeneration can be utilized to calculate the total grams of carbon removed per gram of catalysts. Integration of the carbon curves shown in FIG. 4 gives a total volume of paraffin emitted during the regeneration which can be converted to grams of carbon removed from the catalyst as shown below.

$$Methane \text{:} 3931 mL \times \frac{1 L}{1000 mL} \times \frac{1 mol}{24.6 {L}^{\to }} \times \frac{1 mol C}{1 mol {CH}_{4}} \times \frac{12 g C}{1 mol C} = 2.0 g C$$

$$Ethane \text{:} 504 mL \times \frac{1 L}{1000 mL} \times \frac{1 mol}{24.6 {L}^{←}} \times \frac{2 mol C}{1 mol {C}_{2} {H}_{6}} \times \frac{12 g C}{1 mol C} = 0.5 g C \\ * 24.6 L is the volumetric to molar relationship at 25 ° C . and 1 atm$$

When the regeneration is run to maximize system performance, the amount of carbon per gram of catalyst can be utilized to determine average rate of deposit for carbonaceous species as well as provide some predictive information on the duration between regenerations assuming similar operating conditions are used.

The following examples are included solely to provide a more complete disclosure of the subject invention. Thus, the following examples serve to illuminate the nature of the invention, but do not limit the scope of the invention disclosed and claimed herein in any fashion.

### EXAMPLES

### Example 1

A hydrogenation catalyst regeneration was carried out as follows. Feed was initially switched from sucrose to deionized water to flush soluble components out of the system. The temperature within the catalyst bed was then decreased to less than about 100° C. by turning off electrical heaters in contact with the reactor walls. During the cool down, hydrogen was circulated through the system at a gas hourly space velocity (GHSV) of 500 standard volumes of gas/volume of catalyst/hour using a recycle compressor. A pressure of 1200 psig was maintained on the system. After flushing with more than four reactor volumes of water, the water flow was stopped, the recycle compressor stopped, and the system was depressurized to atmospheric pressure.

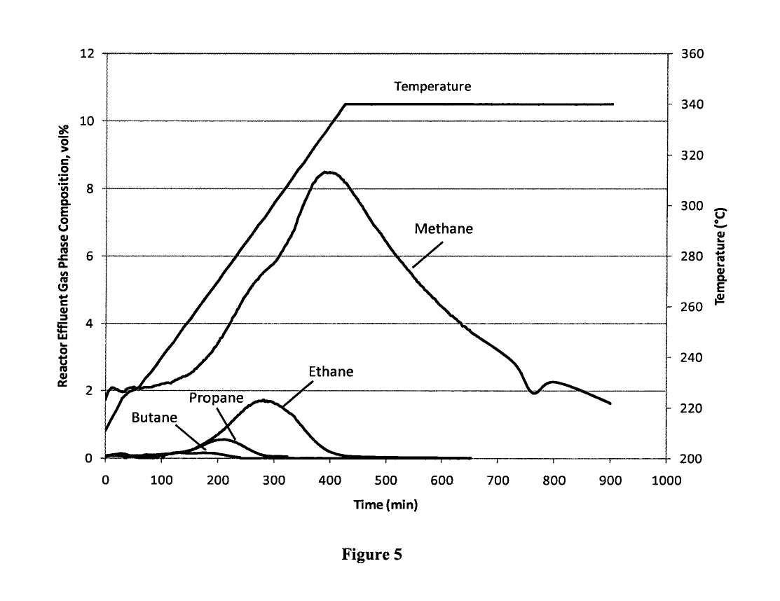

A flow of hydrogen was maintained across the catalyst bed during the depressurization and further cooling to remove adsorbed water from the catalyst. The system pressure was then brought up to 1000 psig using hydrogen and the reactor temperature was increased to 200° C. in approximately 1 hour. The recycle compressor was restarted and total hydrogen flow established at a GHSV of approximately 600 standard volumes of gas/volume of catalyst/hour. A purge of hydrogen equal to approximately 20% of the total flow was removed from the system and analyzed by GC. Pressure on the system was maintained by adding sufficient hydrogen. With hydrogen flow continuing and the pressure maintained at 1000 psig, the temperature of the reactors were gradually increased to 340° C. at a ramp rate of approximately 20° C./hour and then maintained at 340° C. for approximately 8 hours. The methane, ethane, propane, and butane content of the purge gas during the procedure is shown in FIG. 5. Larger paraffins were also released in smaller quantities. The total carbon removed from the catalyst was equal to around 12% of the initial catalyst weight. At the end of the procedure, the reactor heater set points were reset to the normal operating temperatures. Once the reactor cooled to the desired temperature, the normal operating pressure was established and the sucrose feed restarted.

### Example 2

The procedure of Example 1 was followed except that after maintaining the temperature at 340° C. for eight hours, the temperature was increased to 400° C. to determine if additional carbon would be removed at higher temperatures. Less than 0.1% of the initial catalyst weight in additional carbon was removed between 340° C. and 400° C. This indicates that the regeneration was essentially complete at 340° C.

### Example 3

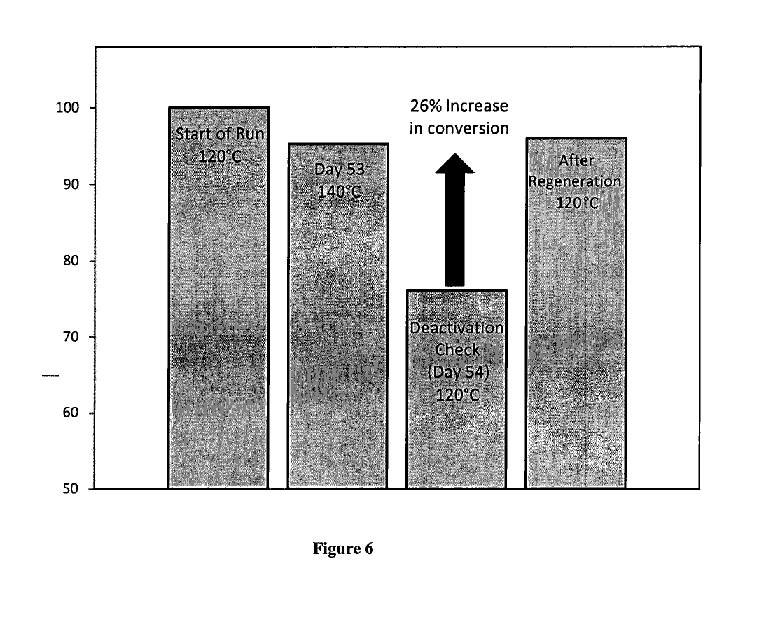

The procedure of Example 1 was followed except that the temperature was ramped to 400° C. and the pressure maintained at 700 psig during the regeneration. The yield of polyols (sorbitol+mannitol) from sucrose before and after the regeneration is shown in FIG. 6. At the same operating condition, the procedure resulted in a 26% increase in conversion for the regenerated catalyst compared to the deactivated catalyst.

### Example 4

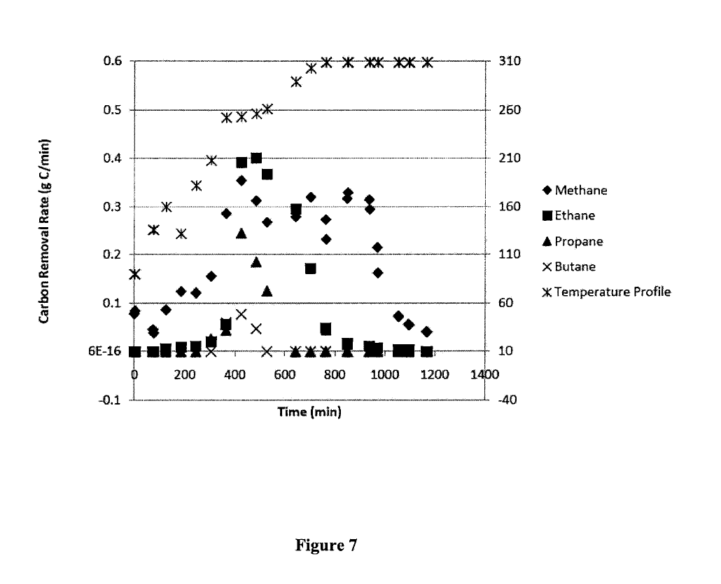

After 130 hours of hydrogenation, the hydrogenation system was shut down, and the catalyst was regenerated using a hot hydrogen strip. During the regeneration hydrogen flow was maintained at approximately 0.16 kg/hr and the temperature was increased to 309° C. over 13 hours. The temperature was maintained at 309° C. for six hours. The effluent gas was sampled and the results are shown in FIG. 7.

As shown in FIG. 7, methane was the dominant species released, accounting for nearly 59% of the 412 grams of carbon burned off in the regeneration. Ethane, propane, and butane accounted for 29, 9, and 2% of the total carbon, respectively. Light paraffins, including ethane, propane, and butane also evolved with the longer chain species that were released at lower temperatures.

### Example 5

The accumulation of paraffins during regeneration was utilized to calculate the total grams of carbon removed per gram of catalysts. Integration of the carbon curves shown in FIG. 3 gave a total volume of paraffin emitted during the regeneration which was converted to grams of carbon removed from the catalyst as shown below.

$$Methane \text{:} 3931 mL \times \frac{1 L}{1000 mL} \times \frac{1 mol}{24.6 {L}^{\to }} \times \frac{1 mol C}{1 mol {CH}_{4}} \times \frac{12 g C}{1 mol C} = 2.0 g C$$

$$Ethane \text{:} 504 mL \times \frac{1 L}{1000 mL} \times \frac{1 mol}{24.6 {L}^{←}} \times \frac{2 mol C}{1 mol {C}_{2} {H}_{6}} \times \frac{12 g C}{1 mol C} = 0.5 g C \\ * 24.6 L is the volumetric to molar relationship at 25 ° C . and 1 atm$$

With a catalyst loading of 20.7 g, a total of 11 wt % carbon was removed from the system.

We claim:

1. A method for regenerating a hydrogenation catalyst comprising:
providing a hydrogenation catalyst containing carbonaceous deposits; flushing the hydrogenation catalyst with a flushing medium;
contacting the hydrogenation catalyst with hydrogen;
maintaining a flow of hydrogen over the hydrogenation catalyst;
adjusting the pressure on the hydrogenation catalyst to a regeneration pressure of about atmospheric pressure to about 3000 psig;
adjusting the temperature of the hydrogenation catalyst to a regeneration temperature in the range of about 250° C. to about 400° C.;
wherein carbonaceous deposits are removed from the hydrogenation catalyst and the hydrogenation catalyst is regenerated such that hydrogenation can be resumed.

2. The method of claim 1 wherein the hydrogenation catalyst is flushed with the flushing medium at a flushing temperature below about 100° C.

3. The method of claim 1 wherein the flushing medium is in the liquid phase.

4. The method of claim 1 wherein the temperature of the hydrogenation catalyst is adjusted to the regeneration temperature at a rate of about 20° C. per hour to about 100° C. per hour.

5. The method of claim 1 wherein the regeneration temperature is maintained for at least about eight hours.

6. The method of claim 1 wherein the regeneration pressure is in the range of about 600 psig to about 1500 psig.

7. The method of claim 1 wherein about 98% of the carbonaceous deposits are removed from the hydrogenation catalyst.

8. The method of claim 1 wherein the flushing medium is selected from the group consisting of water, an alcohol, a ketone, a cyclic ether, a water-soluble oxygenated hydrocarbon, and a combination of any two or more of the foregoing.

9. The method of claim 1 wherein the hydrogenation catalyst is flushed in the presence of hydrogen to maintain an oxygen-free environment.

10. The method of claim 1 wherein the hydrogenation catalyst comprises a support and a catalytic member selected from the group consisting of Fe, Ru, Os, Ir, Co, Rh, Pt, Pd, Ni, Re, Cu, an alloy of at least two of the foregoing, and a combination of at least two of the foregoing.

11. The method of claim 10 wherein the hydrogenation catalyst further comprises a second catalytic material selected from the group consisting of Ag, Au, Cr, Zn, Mn, Sn, Bi, Mo, W, B, P, an alloy of at least two of the foregoing, and a combination of at least two of the foregoing.

12. The method of claim 10 wherein the support comprises a member selected from the group consisting of a nitride, carbon, silica, alumina, zirconia, titania, vanadia, ceria, boron nitride, heteropolyacid, kieselguhr, hydroxyapatite, zinc oxide, chromia, and a combination of at least two of the foregoing.

13. The method of claim 10 wherein the support is a carbon support and the hydrogenation catalyst is flushed in the presence of hydrogen to maintain an oxygen-free environment.

14. A method for hydrogenation of a sugar and in-line regeneration of a hydrogenation catalyst that contains carbonaceous deposits comprising:
catalytically reacting in a liquid or vapor phase an aqueous solution comprising water and a sugar with hydrogen in the presence of the hydrogenation catalyst at a hydrogenation temperature and a hydrogenation pressure;
replacing the aqueous solution with a flushing medium;
contacting the hydrogenation catalyst with hydrogen;
maintaining a flow of hydrogen over the hydrogenation catalyst;
adjusting the pressure on the hydrogenation catalyst to a regeneration pressure in the range of about atmospheric pressure to about 3000 psig;
adjusting the temperature of the hydrogenation catalyst to a regeneration temperature in the range of about 250° C. to about 400° C. and wherein the carbonaceous deposits are removed from the hydrogenation catalyst and the hydrogenation catalyst is regenerated such that hydrogenation can be resumed;
returning the hydrogenation catalyst to the hydrogenation temperature and the hydrogenation pressure; and
catalytically reacting the aqueous solution with hydrogen in the presence of the hydrogenation catalyst at the hydrogenation temperature and the hydrogenation pressure.

15. The method of claim 13 wherein the hydrogenation catalyst is flushed with the flushing medium at a flushing temperature below about 100° C.

16. The method of claim 13 wherein the flushing medium is in the liquid phase.

17. The method of claim 13 wherein the temperature of the hydrogenation catalyst is adjusted to the regeneration temperature at a rate of about 20° C. per hour to about 100° C. per hour.

18. The method of claim 13 wherein the regeneration temperature is maintained for at least about eight hours.

19. The method of claim 13 wherein the regeneration pressure is in the range of about 600 psig to about 1500 psig.

20. The method of claim 13 wherein about 98% of the carbonaceous deposits are removed from the hydrogenation catalyst.

21. The method of claim 13 wherein the flushing medium is selected from the group consisting of water, an alcohol, a ketone, a cyclic ether, a water-soluble oxygenated hydrocarbon, and a combination of at least two of the foregoing.

22. The method of claim 13 wherein the hydrogenation catalyst is flushed in the presence of hydrogen to maintain an oxygen-free environment.

23. The method of claim 13 wherein the hydrogenation catalyst comprises a support and a catalytic material selected from the group consisting of Fe, Ru, Os, Ir, Co, Rh, Pt, Pd, Ni, Re, Cu, an alloy of at least two of the foregoing, and a combination of at least two of the foregoing.

24. The method of claim 23 wherein the hydrogenation catalyst further comprises a second catalytic material selected from the group consisting of Ag, Au, Cr, Zn, Mn, Sn, Bi, Mo, W, B, P, an alloy of at least two of the foregoing, and a combination of at least two of the foregoing.

25. The method of claim 23 wherein the support comprises a member selected from the group consisting of a nitride, carbon, silica, alumina, zirconia, titania, vanadia, ceria, boron nitride, heteropolyacid, kieselguhr, hydroxyapatite, zinc oxide, chromia, and a combination of at least two of the foregoing.

26. The method of claim 23 wherein the support is a carbon support and the hydrogenation catalyst is flushed in the presence of hydrogen to maintain an oxygen-free environment.
